(19) United States
(12) Patent Application Publication
(10) Pub. No.: US 2010/0137769 A1
(43) Pub. Date: Jun. 3, 2010

(54) Adjustable, reusable, disposable finger splint to restrict flexion of the phalangeal joints of the hand

(75) Inventor: Stephen J. Schulte, Marshfield, WI (US)

Correspondence Address: Stephen John Schulte, M220 Sugarbush Lane, Marshfield, WI, 54449, US

(73) Assignee: Baaron John Schulte, New York, NY (US)

(21) Appl. No.: 12/315,107

(22) Filed: Nov. 28, 2008

(51) Int. Cl. A61F 5/00

(52) U.S. Cl. 602/22

(57) ABSTRACT

This invention relates generally to a device that can restrict the flexion of the joints of the fingers. It is primarily used in the treatment of trigger finger and trigger thumb but is effective in any circumstance where the need to restrict flexion of a finger joint is desired. The device is adjustable, reusable and disposable. It consists primarily of a flat strip of material with an adhesive segment at the end that can be rolled into a restraining tubular sleeve around the affected digit. The placement is adjustable according to the individual, unique requirements of the treatment and level of comfort desired by the user.

### REFERENCES CITED TO RELATED APPLICATIONS

### U.S. Patent Documents

| | | | | |
|---|---|---|---|---|
| 3,170,460 | February 1965 | Stilson | 602/22 | |
| 4,243,026 | January 1981 | Barber | 602/22 | 128/87A |
| 4,270,528 | June 1981 | Hanson | 602/22 | |
| 4,297,992 | November 1981 | LaRue et al. | 602/22 | 434/166 |
| 4,441,489 | April 1984 | Evans et al. | 602/22 | 128/77 |
| 4,674,487 | June 1987 | Schaeffer | 602/22 | 128/87A |
| 4,770,166 | September 1988 | Garris | 602/22 | |
| 4,932,396 | June 1990 | Garris | 602/22 | 29/896 |
| 5,267,945 | December 1993 | Doctor etal | 602/22 | |
| 5,197,943 | March 1993 | Link | 602/22 | |
| 5,520,626 | May 1996 | Schaeffer | 602/22.5 | 2/21 |
| 6,110,136 | August 2000 | Belkin | 602/22 | |

### STATEMENT OF FEDERALLY SPONSORED RESEARCH OR DEVELOPMENT

Not Applicable

### NAMES OF JOINT RESEARCH PARTIES

Not Applicable

### REFERENCE TO “SEQUENCE LISTING”

Not Applicable

### BACKGROUND OF INVENTION

(1) Field of the Invention

US Classification 602/22

This invention relates generally to a device that can restrict the flexion of the joints of the fingers. It is primarily used in the treatment of trigger finger and trigger thumb but is effective in any circumstance where the need to restrict flexion of a finger joint is desired. The device is adjustable, reusable and disposable. It consists primarily of a flat strip of material with an adhesive segment at the end that can be rolled into a restraining tubular sleeve around the affected digit. The placement is adjustable according to the individual unique requirements of the treatment and level of comfort desired by the user.

(2) Background Art

There are multiple disease processes of the hand and digits that can cause painful ‘locking’ of the finger joints upon flexion. While arthritis and injury can be causative of this condition one of the more common ailments in this category is trigger finger or trigger thumb. Regardless of a specific cause though, the end result is a painful locking of the joint if it is flexed beyond a critical point. Often the only way to extend the joint again is by manual manipulation with the other hand that invariably produces a painful ‘snap’ as the tendon is forcefully straightened.

There are many treatments for trigger finger and other afflictions of the digits and many of them can be curative. Mayo clinic sites the use of aspirin and other pain killers in the acute phase of injury with rest and immobilization of the digit for up to six to eight weeks to allow the injury time to heal. If that is not effective then steroid injections into the offending tendon is tried. Besides being painful and variable in outcome such injections, repeated often enough, may result in rupture of the tendon and further damage. There are many people who are reluctant to face repeated, painful injections and elect on surgery to free the tendon. Surgery may be successful but sometimes it is not and one is then faced with the prospect of the continuing reoccurrence of this painful locking as an unwanted but inevitable fact of life. These problems afflict the elderly and women a disproportionate amount, the very persons who may be on fixed incomes or lack the medical insurance or other funds to be provided choices. In the end, a population of individuals is created that have found no permanent relief. They are faced with continual pain and increased inflammation from the repeated ‘triggering’ of the defective tendons. The recurrent trauma then compounds their problem even more.

I am intimately acquainted with these problems as my wife has had trigger finger for years now. I have watched as she struggled at restaurants when a joint would lock as she grasped a coffee cup and then be faced with the dilemma of trying to release her grip without jerking and spilling the coffee. One has to be on constant alert to flex the joint just enough to do what you want to do but not so much as to trigger the reaction. It is tiring, aggravating and embarrassing. We have searched for an answer for a long time.

It is true that totally immobilizing the offending joint will ‘cure’ the problem. If it were that simple then any number of the rigid splinting devices currently available on the market (and sited in this application) would work. Unfortunately the only devices available to the public at this time are directed at immobilization of the joint by use of bulky metal appendages or rigid rings that slide over the finger. There are inherent problems with all of these devices. Bulky metal splints. as exemplified by U.S. Pat. No.4,243,026, Barber and U.S. Pat. No. 4,441,489, Evans, completely restrict movement and can make the simplest daily tasks aggravating and tiresome, from washing your hands, buttoning shirts, to brushing your teeth. It is difficult to type and navigate the Internet with a metal ‘stick’ for a finger.

The rings that slide onto the fingers must be custom adjusted to the user. And even if you get one that fits, daily swelling and change in the size of the knuckles with arthritis can alter the size of the joint on a daily basis. (see U.S. Pat. Nos. 4,270,528, 4,297,992, 4,674,487, 4,932,396, and 6,110,136 above as well as the device described in Patent Application 20070167894, Ryscavage, which is also rigid as well as sporting a ‘palm pad’ that applies pressure to the ligaments of the hand. Such a rigid device may make gripping of a cane or walker handle difficult and painful if much pressure is applied to the grip and furthermore, its positioning is restricted to the metacarpal-phalangeal joint (MCP) only.) A ring device large enough to slide over an enlarged joint may not fit correctly on the next joint where it is needed. One that fits today may not fit tomorrow. It may fall off and be lost. Faced with possible daily variation of joint size a person would need a vast collection of these devices on hand, of many differing sizes, to assist them on a daily basis. In addition, many persons do not wish to call attention to their ailment by wearing bulky and obvious corrective devices on their hands in public.

What is needed is a device that would be light and not too noticeable. It would restrict the movement of the joint enough to prevent the trigger reaction but not so much as to impair normal daily use of the fingers. It would be versatile enough to be reusable many times but cheap enough to be discarded on a routine basis. The device would be infinitely adjustable, comfortable and adaptable to the individual's needs as well as easy to use.

This invention satisfies these needs.

### SUMMARY OF THE INVENTION

It is the general object of the present invention to restrict the flexing of the finger joints of the hand, primarily, but not limited to the Proximal Interphalangeal (PIP) joint. It is a more particular object of this invention to restrict this motion without unduly restricting the normal fine finger movement needed for daily function. It is a still more particular object of this invention to be lightweight, low cost, easy to use and adjustable to the particular unique needs of each individual.

In accordance with the present invention there is provided a device comprised of a singular strip of plastic, vinyl or similar material approximately one inch wide by four inches long, the last half inch of which is coated with an adhesive on one side. The material is preferred to be transparent and of the size previously indicated but stating so is a matter of example and not limitation of design and function. The strip of material is wrapped around the offending joint in a comfortable manner (Drawing 2, FIGS. 2-4) and then locked into a tubular form when the last portion with the adhesive completes the wrap. The device can be successfully employed in acute cases of injury to aid in the anticipated six to eight week healing process of damaged tendons. The lightweight construction of the device and its unobtrusive function make it more likely a patient would wear it round the clock for the full period of recovery rather than prematurely discontinue use of a less comfortable device. Ultimately though, the primary intended use is to prevent the locking and pain in the joints on a daily basis for persons chronically afflicted with the problem. Those persons to whom a total ‘cure’ has been illusive

It is a further aspect of the present invention to be variable in size in order to accommodate a range of uses. The flexible, conformative nature of the device makes it ideally suited for these problems in that it can be easily modified by the user, through length or thickness variations, to provide ‘just enough’ restriction to the joint to stop the triggering but still allow maximum range of ‘safe’ movement of the joint. The user enjoys the substantial benefits of this superior design by experiencing much less disruption to normal function of the joint in routine daily activities. All of the other, absolutely rigid, devices sited do not support such a variable range but are rather, ‘all or nothing’.

It is a further aspect of the present invention that the current dimension is given as an example and not a limitation of the design and function of the device and that the treatment of trigger finger is used as an example of the device's function but it is not meant to limit its use solely to the treatment of this singular affliction. The device is designed to restrict the flexing of one or more finger joints on one or more fingers regardless of the cause of the infirmary being trigger finger, trigger thumb, arthritis, injury or other.

### BRIEF DESCRIPTION OF THE DRAWINGS

FIG. 1 is a perspective view of the device (1), the area of adhesive coating is indicated by (2)

FIG. 2 illustrates the placement of the finger within the device prior to construction of the final tubular form.

FIG. 3 illustrates the approximate midpoint of the application/construction process

FIG. 4 illustrates the completed construction of the device in place on the finger

FIG. 5 is a representation of the finger position of a typical locked joint illustrating the position of the Distal Interphalangeal Joint (DIP), the ‘locked’ Proximal Interphalangeal joint (PIP) and the Metacarpal-phalangeal joint

FIG. 6 illustrates the corrective placement of the device over the PIP joint with the finger now in normal position.

### DESCRIPTION OF THE PREFERRED EMBODIMENT

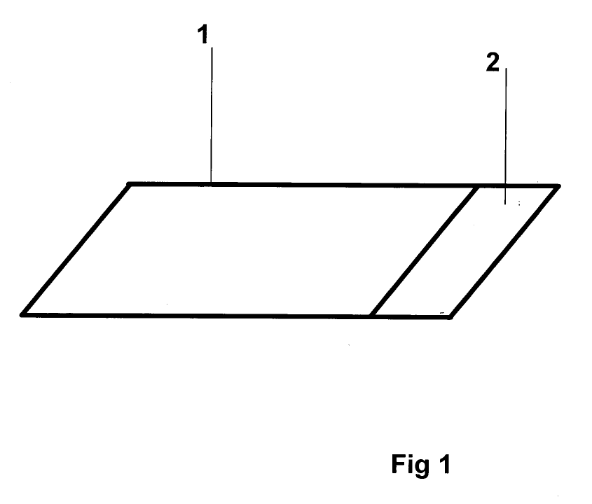

FIG. 1 is an overall view of the present invention. The overall length is 4 inches. The width is 1 inch. The last one-half inch of the material (2) is coated with a pressure sensitive adhesive. The device is constructed of soft vinyl plastic that is 12 mil (0.30 mm.) in thickness and clear in coloration with a matte finish. This material is highly conformable and resistant to aging, sun, water, fungus, bacteria, oil, acids, alkalis and corrosive chemicals. It provides good abrasion, impact and rupture resistance with a tensile strength of 16 lbs./in and elongation at break point of 150%. The adhesive is synthetic rubber resin, applied to one side of the distal one-half inch of the device (2).

The vinyl material is lightweight and easily shaped into the tubular form. At this thickness (0.3 mm) it is rigid enough to adequately restrict the flexion of the PIP joint to prevent the ‘triggering’ of the tendon but still pliable and flexible enough to be comfortably worn for long periods of time. This is especially important when the device is worn overnight to prevent the locking of one or more joints and the painful straightening that occurs in the morning. Additionally, the material is easily cut with scissors to further customize the fit to the unique needs of the individual. The width may be reduced to better fit a smaller finger and the length may also be reduced to accommodate the user either before or after the construction of the tubular form.

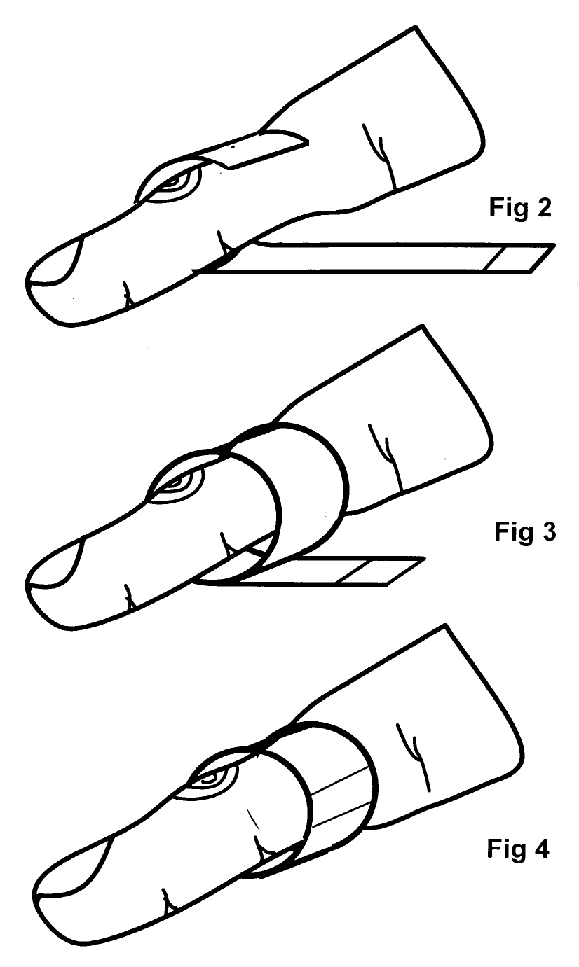

FIGS. 2-4 illustrate the method of constructing the tubular form to fit the finger. This is the feature that allows infinite variability for the user. The device can be wrapped around any portion of the finger. The wrap may be constructed so it is tight or loose depending on the uniqueness of the individual's wants and the severity of the affliction. It is easy to construct a tube that is adequate to prevent the flexion of the joint but still loose enough to be easily removed for hand washing or other needs. It can be trimmed in a number of ways to better match the comfort requirements of the user. Users that require the minimum amount of restraint can shorten the vinyl strip so the final tube is only one layer thick around the finger. If more resistance is needed the thickness of the tube (and its rigidity) can be increased simply by doubling a second strip over the first. The flexibility and easy conformability of the vinyl make constructing the device over joints of varying size easy to do. The lightweight material is easily tolerated by users of all ages and the transparent color allows relief in all social settings without calling needless attention to the affliction.

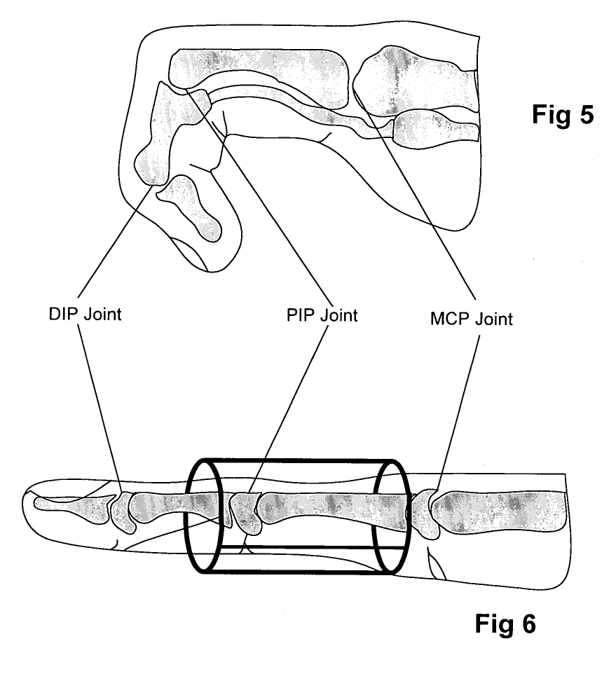

FIG. 5 shows alignment of the bones and joints when a digit is in a locked position.

FIG. 6 shows the correct positioning of the device on the finger. It is desirable to find a comfortable location for wearing the device so that it extends just distal (toward the fingertip) to the PIP joint. The exact location for optimal performance will vary with the user but in general, the more the device extends distal to the PIP joint the less flexibility allowed the PIP joint. For some users, even modest flexion of the PIP joint will precipitate a locking. These users will find maximum relief if the PIP joint is more greatly restricted by an extension of the device more distal to the joint as well as increased thickness (rigidity) of the constructed tube. The goal, of course, is to allow the greatest flexibility and use of the fingers while preventing the locking of the PIP.

Each individual will find that ideal placement with just a modest amount of experimenting by sliding the device forward and backward until the best location is ascertained. Such placement allows maximum dexterity of the DIP and PIP joints and allows typing, needlework and other fine motor skills to be exercised to their greatest extent.

Although the device is designed to be used on the digits of the hand it is possible to adapt it for the toes, in the event that flexing of the toe joints needs to be restricted. The size of the device would be altered to accommodate the smaller digits to provide the same function and therapeutic effect as the primary device.

While the present invention has been described in terms of specific embodiments thereof, it will be understood that no limitations are intended to the details of construction or design or practice of the invention other than as defined in this document.

What is claimed is:

1. An adjustable, reusable, and disposable device to restrict the flexion of the Proximal Interphalangeal Joint (PIP) of the finger to avoid locking of said joint caused by, but not limited to, the afflictions known as trigger thumb and trigger finger as well as causes arising from the complications of injury or arthritis to the hands and fingers, comprising a strip of material having pressure sensitive adhesive coated on one side of the last one-half inch of its length and able to be rolled and conformed by the user into a tubular shape that overlays and encircles the PIP joint so as to restrict the flexion thereof.

2. A device as defined in claim 1, wherein the preferred material is comprised of soft vinyl plastic, clear in coloration.

3. A device as defined in claim 1, wherein comprised of material with the preferred thickness of 12 MIL (0.3 mm)

4. A device as defined in claim 1, wherein comprised with preferred strip dimensions of 1 inch wide and four inches long.

5. A device as defined in claim 1, wherein comprised with preferred pressure sensitive synthetic rubber resin adhesive coating to the distal one-half inch of the strip

6. A device as defined in claim 2, wherein comprised of an alternative, suitably pliable and rigid material including but not limited to vapor permeable films, microporous films, plastic films, polymers, fibrous materials and pulp fibers either single or multi-layered in composition and of any color or opacity or decoration.

7. A device as defined in claim 3, wherein comprised of material with variable alternative thickness' not less than 1 MIL (0.025 mm) nor greater than 36 MIL (0.9 mm)

8. A device as defined in claim 4, wherein comprised of alternative strip dimensions variable from one-half to three inches wide and two to eight inches in length and all combinations thereof.

9. A device as defined in claim 5, wherein comprised with an alternative pressure sensitive adhesive of natural or synthetic origin coating the distal portion of the strip.

10. A device as defined in claim 1, wherein comprised of preformed tubular structures of various sizes.
